(19) United States
(12) Patent Application Publication
(10) Pub. No.: US 2024/0156552 A1
(43) Pub. Date: May 16, 2024

(54) SURGICAL ROBOT

(71) Applicant: SONY GROUP CORPORATION, TOKYO (JP)

(72) Inventor: HIROYUKI SUZUKI, TOKYO (JP)

(21) Appl. No.: 18/551,404

(22) PCT Filed: Dec. 28, 2021
(86) PCT No.: PCT/JP2021/048999

§ 371 Date: Sep. 20, 2023

(30) Foreign Application Priority Data
Mar. 31, 2021 (JP) 2021-059921

(51) Int. Cl. A61B 34/30; B25J 17/00

(52) U.S. Cl. CPC A61B 34/30; B25J 17/00

(57) ABSTRACT

Provided is a surgical robot that is applied to ophthalmic surgery and achieves minimally invasive surgery by reducing a load applied to a portion of insertion by a surgical tool. The surgical robot is configured by a plurality of links, at least some links connecting between links are formed by a flexible joint having flexibility, and a surgical tool is mounted at a distal end. The flexible joint that is a passive joint has a spring element made by, for example, polyimide, CFRP, GFRP, or polyester, in a rotation direction. Furthermore, the flexible joint that is an active joint is connected to a flexible structure that is serially fixed to an output shaft of a driving motor.

### TECHNICAL FIELD

The technology disclosed in this specification (hereinafter, “the present disclosure”) relates to a surgical robot that supports a surgical tool at a distal end in a multi-link structure.

### BACKGROUND ART

Recently, a robotics technology has been introduced into a medical field as well, and surgery is performed safely and accurately using a master-slave surgical system (see, for example, Patent Document 1). By incorporating the robotics technology into the surgical system, it becomes possible to suppress tremor of hands of a surgeon, to assist an operation, to absorb a difference in skill between surgeons, to perform surgery from a remote place, and the like. In general, in a surgical robot, a surgical tool is rigidly supported at a distal end of a robot arm having a multi-link structure. Therefore, in a situation where a plurality of surgical tools is inserted into one surgical site (for example, the same eyeball), when one surgical tool moves, a load is directly applied to a portion of insertion by another surgical tool.

In fundus surgery, a surgical tool is inserted into an eyeball via a mantle tube called a trocar. Although the trocar is inserted into a sclera (white of the eye) of an eyeball, a load applied to the portion of insertion increases when the surgical tool is operated or the eyeball moves. Therefore, from the viewpoint of minimal invasiveness, it is desirable to reduce the load applied to the portion of insertion.

### CITATION LIST

### Patent Document

Patent Document 1: WO2019/012812

### SUMMARY OF THE INVENTION

### Problems to be Solved by the Invention

An object of the present disclosure is to provide a surgical robot that is mainly applied to ophthalmic surgery and achieves minimally invasive surgery by reducing a load applied to a portion of insertion by a surgical tool.

### Solutions to Problems

The present disclosure has been made in view of the problems described above, and is a surgical robot that is configured by a plurality of links and is mounted with a surgical tool, in which at least some links connecting between links are formed by a flexible joint having flexibility.

The flexible joint that is a passive joint has a spring element in a rotation direction. The spring element is, for example, any of polyimide, CFRP, GFRP, and polyester.

Alternatively, the flexible joint that is an active joint is connected to a flexible structure that is serially fixed to an output shaft of a driving motor.

### Effects of the Invention

According to the present disclosure, it is possible to provide a surgical robot that is mainly applied to ophthalmic surgery and achieves minimally invasive surgery by reducing a load applied to a portion of insertion by a surgical tool.

Note that the effects described in this specification are merely examples, and the effects brought by the present disclosure are not limited thereto. Furthermore, the present disclosure may further provide additional effects in addition to the effects described above.

Still another object, feature, and advantage of the present disclosure will become clear by further detailed description with reference to an embodiment to be described below and the attached drawings.

### BRIEF DESCRIPTION OF DRAWINGS

FIG. 1 is a view illustrating a configuration example of a surgical robot 100.

FIG. 2 is a view illustrating a layout of fundus surgery using the surgical robot 100.

FIG. 3 is a view illustrating a configuration example of a robot 300 that easily pivotally moves.

FIG. 4 is a view illustrating an operation example of the robot 300.

FIG. 5 is a view illustrating a cross-sectional configuration example of an electric circuit board 500.

FIG. 6 is a view illustrating a configuration example of an open link structure body 600 configured using an FPC.

FIG. 7 is a view illustrating an example of a closed link structure body 700 configured using an FPC.

FIG. 8 is a view illustrating a configuration example of a surgical robot 800 using the FPC.

FIG. 9 is a view illustrating a three-dimensional image example of the surgical robot 800 using the FPC.

### MODE FOR CARRYING OUT THE INVENTION

Hereinafter, the present disclosure will be described in the following order with reference to the drawings.
A. Overview
B. Configuration of surgical robot
C. Layout of fundus surgery
D. Configuration of pivotally movable robot
E. Configuration method for multi-link structure body

A. Overview

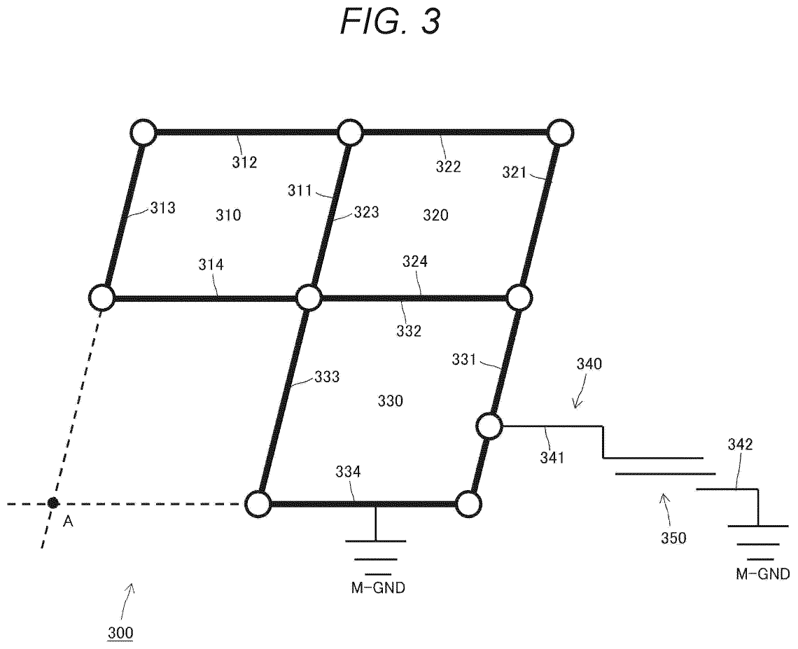

For example, FIG. 3 of Patent Document 1 describes a surgical robot used on a slave side in a master-slave surgical system. In general, this type of surgical robot is constituted by a robot arm having a multi-link structure, and a surgical tool such as a forceps is mounted at a distal end thereof. The forceps is inserted into a surgical site via a mantle tube such as a trocar. Therefore, from the viewpoint of minimal invasiveness, it is desirable to reduce a load applied to a portion of insertion by the surgical tool.

Here, a case where the surgical robot is applied to fundus surgery will be considered. In a case where the robot arm is made by a rigid body such as a metal material, and the robot arm is rigidly fixed to a mechanical ground (such as an operation table), a load applied to the portion of insertion by the trocar increases when the surgical tool is operated or the eyeball moves.

Furthermore, in a case where a plurality of surgical tools is inserted into the same eyeball and each surgical tool is rigidly fixed to the robot arm, when one surgical tool moves, a load is directly applied to a portion of insertion by another surgical tool. In order to reduce the load applied to the portion of insertion, it is ideal that the surgical tool pivotally moves at the portion of insertion. In a case where an error occurs with respect to an ideal pivot movement, when one surgical tool moves, a load applied to a portion of insertion by another surgical tool cannot be ignored.

In general, as the robot arm becomes larger, a kinematic error increases due to deflection caused by its own weight. For example, an error occurs on the order of several centimeters in an industrial robot. Furthermore, in a case where a plurality of robot arms is fixed to the operation table, an error caused by an alignment error of each robot arm occurs.

Therefore, in the present disclosure, in a robot arm having a multi-link structure, a spring element, that is, a “flexible joint” having flexibility in a rotation direction is used as a joint that hinge-connects between links. Therefore, according to the present disclosure, even in a case where an error occurs in a pivot movement with respect to an ideal at a portion of insertion when the robot arm operates a surgical tool, or when the eyeball moves or when another surgical tool inserted into the same eyeball is operated, the error and the movement of the eyeball are absorbed by the flexible joint, a load at the portion of insertion is reduced, and minimally invasive surgery can be achieved.

Note that, in a case where positioning control of a tip end position of the surgical tool is performed using the robot arm including the flexible joint, a rotation angle of the joint is displaced from a position command value by an external force acting on the surgical tool or the like. Even in such a case, it is possible to measure a displacement amount from the position command value by arranging an encoder in each flexible joint, and it is possible to maintain positioning accuracy of the tip end of the surgical tool by correcting the position command value.

Also by making a link flexible instead of the joint connecting between links, an external force can be absorbed and minimally invasive surgery can be achieved. However, while a displacement amount of the joint can be easily measured using the encoder, it is difficult to measure a displacement amount (deflection) of the link during surgery. Therefore, from the viewpoint of maintaining the positioning accuracy of the tip end of the surgical tool, it is possible to achieve highly accurate and minimally invasive surgery by configuring the surgical robot by using the flexible joint instead of the flexible link.

B. Configuration of Surgical Robot

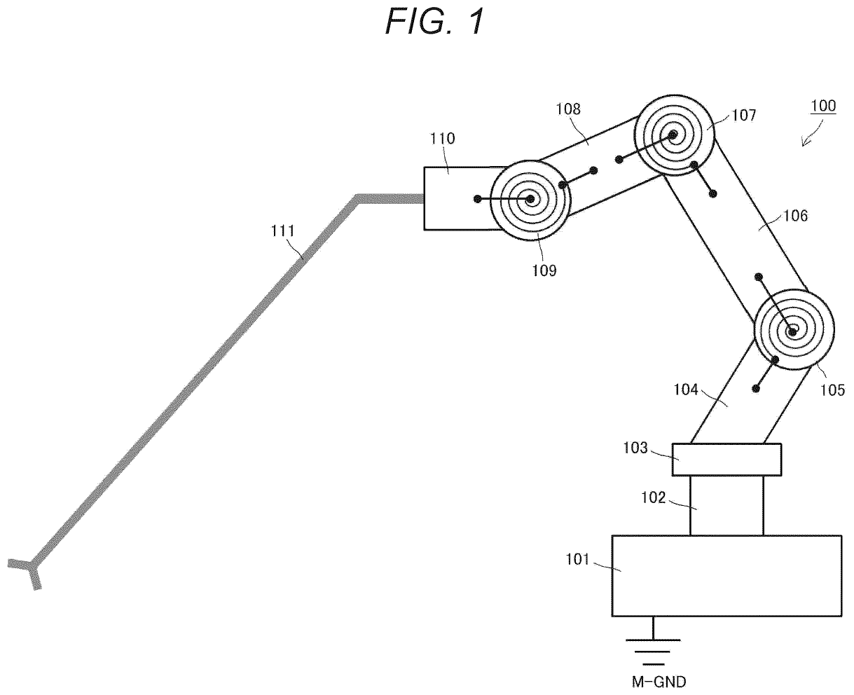

FIG. 1 illustrates a configuration example of a surgical robot 100 to which the present disclosure is applied. The illustrated surgical robot 100 includes a base part 101 rigidly fixed to a mechanical ground (M-GND), a link 102 attached perpendicularly to the base part 101, and a robot arm attached to an upper end of the link 102 via a joint 103. The joint 103 is assumed to have a degree of rotational freedom about a yaw axis.

In the example illustrated in FIG. 1, the robot arm has a serial link structure, and includes links 104, 106, 108, and 110, a flexible joint 105 that hinge-connects between the link 104 and the link 106, a flexible joint 107 that hinge-connects between the link 106 and the link 108, and a flexible joint 109 that hinge-connects between the link 108 and the link 110. Then, a surgical tool 111 is mounted to the link 110 at a distal end. In the example illustrated in FIG. 1, each of the flexible joints 105, 107, and 109 has a degree of rotational freedom about a roll axis (alternatively, about an axis orthogonal to the yaw axis).

Here, it is assumed that each of the links 102, 104, 106, 108, and 110 is made by a rigid body. If each of the flexible joints 105, 107, and 109 is a passive joint, a spring element is incorporated in a rotational direction to give flexibility. The spring element is made by, for example, polyimide, carbon fiber reinforced plastic (CFRP), glass fiber reinforced plastic (GFRP), polyester, or the like. Furthermore, if each of the flexible joints 105, 107, and 109 is a drive joint, a flexible structure (or a spring element) serially fixed to an output shaft of a motor is connected thereto. It is preferable that the encoder that measures a displacement amount of the joint is attached to each of the flexible joints 105, 107, and 109, regardless of either the passive joint or the drive joint. Note that the joint 103 may or may not have flexibility.

C. Layout of Fundus Surgery

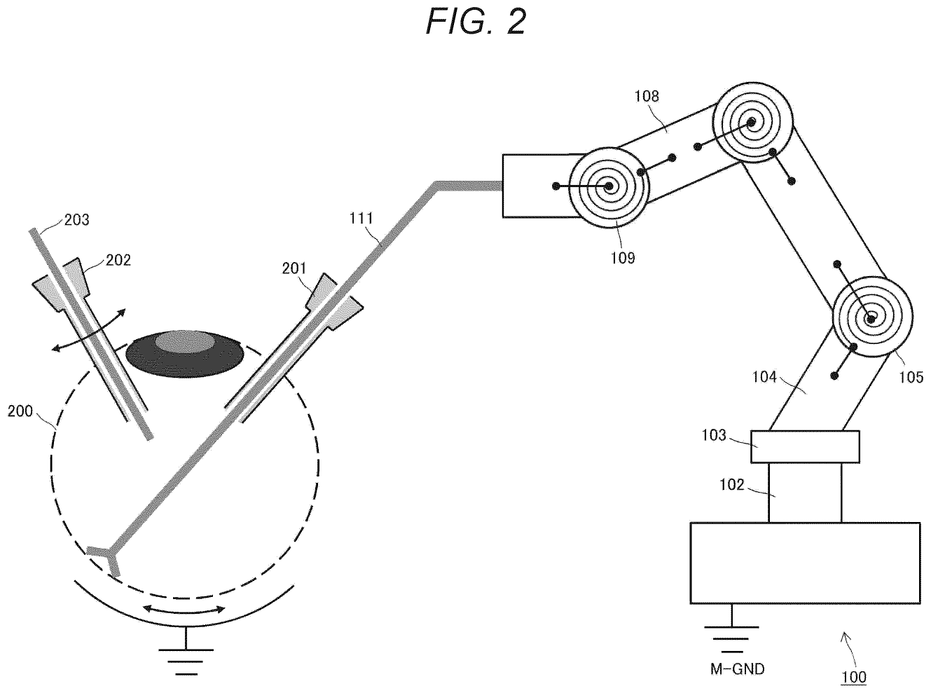

FIG. 2 illustrates a layout of fundus surgery (retinal surgery or the like) using the surgical robot 100 illustrated in FIG. 1. An eye lid speculum (not illustrated) is attached to an eyeball 200 so that the eyelid is not closed. Then, trocars 201 and 202 are inserted into a plurality of locations (two locations in the example illustrated in FIG. 2) on a surface of the eyeball 200.

FIG. 2 illustrates a cross section of the eyeball 200 cut such that the trocars 201 and 202 pass through. The surgical tool 111 mounted at a distal end of the surgical robot 100 is inserted into the eyeball 200 via one trocar 201. Furthermore, another surgical tool 203 is inserted into the eyeball 200 via another trocar 202. It is assumed that the surgical tool 203 is operated by a surgical robot of the same type as or a different type from the surgical robot 100, or is operated by a procedure of a surgeon.

In order to reduce a load applied to the eyeball 200 when the surgical robot 100 is driven to operate the surgical tool 111, it is ideal that the surgical tool 111 pivotally moves at an insertion point of the trocar 201. In a case where an error occurs with respect to an ideal pivot movement, the load applied to the eyeball 200 at the insertion point of the trocar 201 by the operation of the surgical tool 111 cannot be ignored. On the other hand, according to the present disclosure, the surgical tool 111 is supported by the robot arm having the multi-link structure serially connected using the flexible joints 105, 107, and 109. Therefore, even if an error occurs in the pivot movement of the surgical tool 111, the error can be absorbed by displacement of the flexible joints 105, 107, and 109. Therefore, a load applied to the eyeball 200 can be reduced, and minimally invasive surgery can be achieved.

Furthermore, usually, the eyeball 200 can move with respect to a head part (alternatively, a mechanical ground). The movement of the eyeball 200 applies a load to the eyeball 200 at the insertion point of the trocar 201. On the other hand, according to the present disclosure, the surgical tool 111 is supported by the robot arm having the multi-link structure serially connected using the flexible joints 105, 107, and 109. Therefore, an error of the movement of the eyeball 200 during fundus surgery can be absorbed by displacement of the flexible joints 105, 107, 109. Therefore, a load applied to the eyeball 200 can be reduced, and minimally invasive surgery can be achieved.

Furthermore, when the surgical tool 203 is operated by a surgical robot of the same type as or a different type from the surgical robot 100, or is operated by a procedure of the surgeon, when an error occurs in a movement of the surgical tool 203 with respect to an ideal pivot movement, a load is applied and the eyeball 200 moves. Therefore, a load is applied to the eyeball 200 at an insertion point of the trocar 201. On the other hand, according to the present disclosure, the surgical tool 111 is supported by the robot arm having the multi-link structure serially connected using the flexible joints 105, 107, and 109. Therefore, an error of the movement of the eyeball 200 can be absorbed by displacement of the flexible joints 105, 107, 109. Therefore, a load applied to the eyeball 200 can be reduced, and minimally invasive surgery can be achieved.

As described above, according to the surgical robot 100 to which the present disclosure is applied, it is possible to obtain a buffering effect by displacement of the flexible joints 105, 107, and 109 when a load is instantaneously applied to the portion of insertion in the eyeball 200. Therefore, it is possible to reduce complications such as laceration of the portion of insertion in the eyeball 200.

D. Configuration of Pivotally Movable Robot

In the above description, it has been mentioned that the surgical tool should pivotally move at an insertion point in order to reduce a load applied to a portion of insertion in the eyeball. In this Paragraph D, a configuration example of a robot including a mechanism that easily achieves the pivot movement at the insertion point will be described.

FIG. 1 has illustrated the surgical robot 100 having an open link structure for convenience of description. On the other hand, FIG. 3 illustrates a degree of freedom configuration of a robot 300 having a closed link structure, as a configuration of a robot that easily pivotally moves. However, in FIG. 3, a highly rigid link is drawn by a thick line, and a joint to hinge-connect between links is indicated by a circle coaxial with a rotation axis. At least some joints are configured as a flexible joint.

The robot 300 is configured by connecting three closed link structure bodies 310, 320, and 330 in order from a distal end. Among them, one link 334 of the closed link structure body 330 on a proximal end side serves as a mechanical ground (alternatively, a fixed link).

The closed link structure body 310 is a four-node link mechanism including four links 311 to 314, and lengths of facing links are equal to each other. Furthermore, the closed link structure body 320 is a four-node link mechanism including four links 321 to 324, and lengths of facing links are equal to each other. Furthermore, the closed link structure body 330 is a four-node link mechanism including four links 331 to 334, and lengths of facing links are equal to each other. However, the link 312 and the link 322, the link 314 and the link 324, and the link 321 and the link 331 are individually linearly connected to operate as one link. Furthermore, the link 311 and the link 323 that join the closed link structure body 310 and the closed link structure body 320 integrally operate as one link, and the link 324 and the link 332 that join the closed link structure body 320 and the closed link structure body 330 integrally operate as one link.

To the link 331 hinge-connected to one end of the link 334, an open link structure body 340 is connected. The open link structure body 340 includes two links 341 and 342 linearly connected via a linear motion actuator 350. Then, one end of one link 342 is rigidly fixed to a mechanical ground. When the linear motion actuator 350 is operated and the link 341 is displaced in a horizontal direction (or an x direction) on a plane of the figure, the link 331 rotates about a connection point between with the link 334. Therefore, the closed link structure body 330 operates such that the link 331 is a driving link, the link 333 facing the link 331 is a driven link, and another link 332 is an intermediate link, and an angle of facing links is maintained. Then, this operation is transferred to the adjacent closed link structure body 320 and further to the closed link structure body 310 adjacent to the closed link structure body 320.

The link 313 of the closed link structure body 311 corresponds to a link at a distal end of the robot 300, and constitutes a mounting part of an end effector (not illustrated in FIG. 3) including a surgical tool such as a forceps. In a case where the robot 300 performs a surgical operation by operating the surgical tool mounted at the distal end, for the sake of minimal invasiveness, it is necessary to perform surgery with the smallest load as possible with respect to the vicinity of a trocar into which the surgical tool is inserted. Therefore, it is ideal to perform an operation of making an impulse generated at a trocar insertion point zero by pivotally operating the surgical tool with the trocar insertion point as a fulcrum (alternatively, by fixing the trocar insertion point).

In FIG. 3, an axis of the link (fixed link) 334 of the closed link structure body 330 intersects, at a point A, an axis of the link 313 which is a mounting place of the surgical tool of the closed link structure body 310 at a distal end.

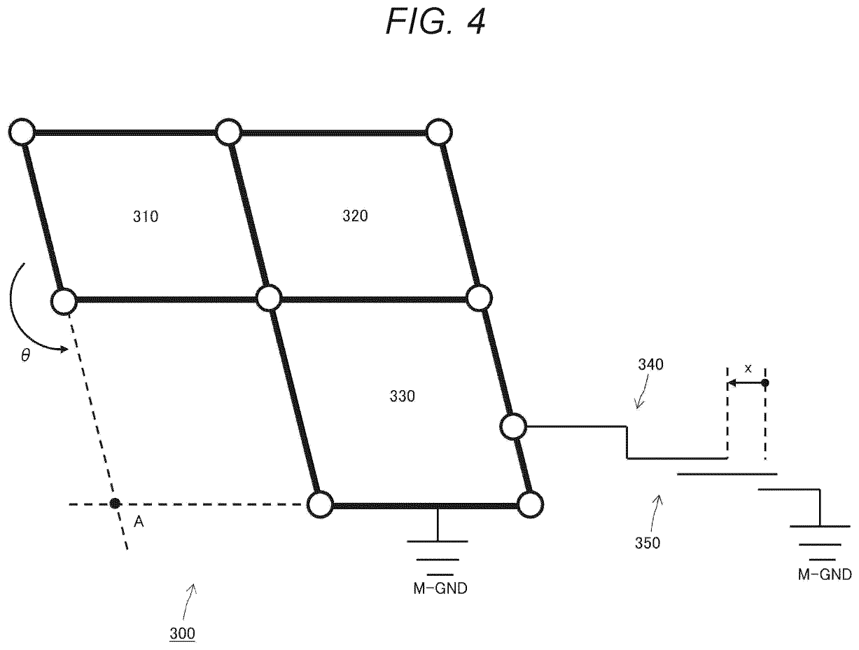

FIG. 4 illustrates a state where the link 331, which is the driving link of the closed link structure body 330, is rotated by an angle θ counterclockwise on a plane of the figure via the open link structure body 340 by displacing the linear motion actuator 350 in an x direction. Assuming that each link of other closed link structure body 320 and closed link structure body 310 maintains a parallel relationship with each corresponding link of the closed link structure body 330, an axis of the link (fixed link) 334 of the closed link structure body 330 similarly intersects, at the point A, an axis of the link 313 which is a mounting place of the surgical tool of the closed link structure body 310 at the distal end. That is, the intersection point A is an immobile point.

Therefore, by setting the intersection point A to be the trocar insertion point, the surgical tool mounted to the link 313 pivotally moves at the portion of insertion, and minimally invasive surgery can be achieved.

Note that a configuration of the robot that achieves the pivot movement is not limited to FIG. 3. Furthermore, the pivot movement may be achieved by motion control of the arm instead of mechanically achieving the pivot movement as illustrated in FIG. 3.

E. Configuration Method for Robot

In this Paragraph E, a description is given to an example of a configuration method for a robot including a multi-link structure having a flexible joint by using a flexible electric circuit board having low rigidity and flexibility.

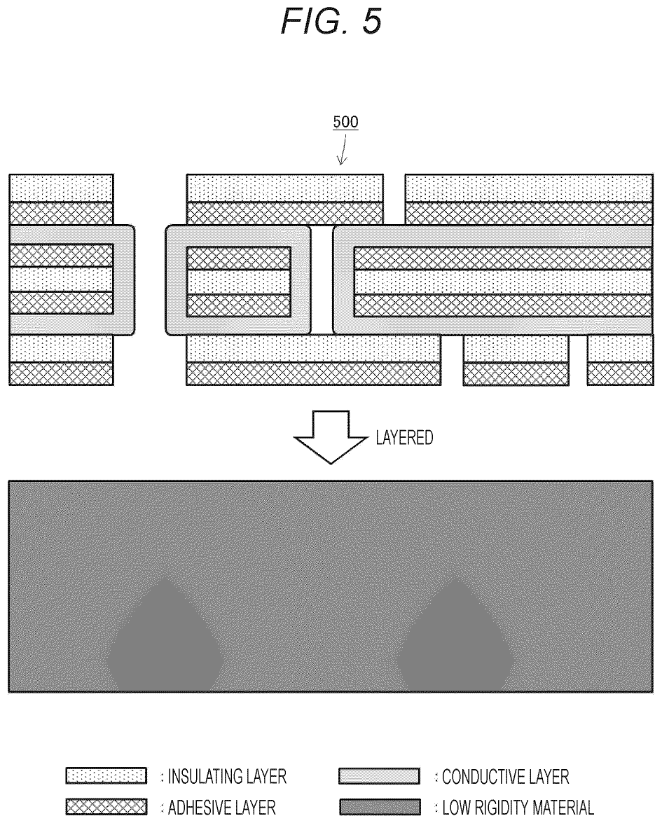

FIG. 5 illustrates a cross-sectional configuration example of an electric circuit board 500 to be applied to a multi-link structure having a flexible joint. As can be seen from the figure, the electric circuit board 500 is a multi-layer structure body including an insulating layer made by a high-electron polymer or polyimide and a conductive layer formed by depositing a metal such as copper or aluminum, and having a plurality of sets of the insulating layer and the conductive layer joined by an adhesive layer. A method for manufacturing the electric circuit board 500 having such a multi-layer structure is not particularly limited. For example, there is also a method of bonding the insulating layer and the conductive layer by providing the adhesive layer on the conductive layer prepared in advance. Then, finally, the multi-layer structure including the insulating layer, the conductive layer, and the adhesive layer is covered with a low rigidity material made by polyimide or the like, so that the electric circuit board 500 having low rigidity and flexibility is achieved. The electric circuit board 500 may be the same as a general flexible printed circuit (FPC).

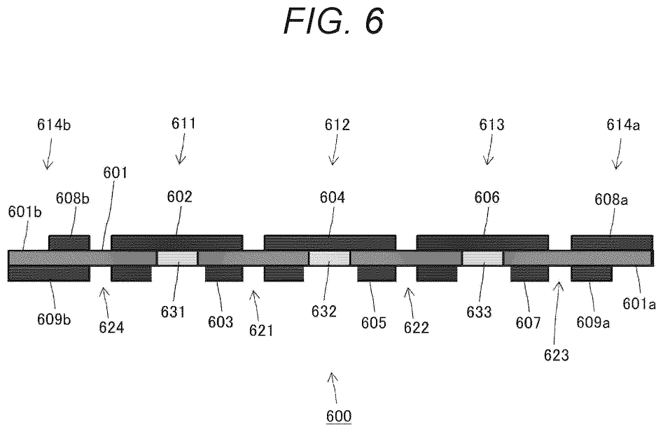

FIG. 6 illustrates a configuration example of an open link structure body 600 configured using an FPC. In the open link structure body 600, a low rigidity FPC 601 is arranged at a center, a pair of strong rigidity portions 602 and 603 are joined to front and back surfaces of the FPC 601 to form a link 611 having rigidity, a pair of strong rigidity portions 604 and 605 are joined to the front and back surfaces of the FPC 601 to form a link 612 having rigidity, and a pair of strong rigidity portions 606 and 607 are joined to the front and back surfaces of the FPC 601 to form a link 613 having rigidity. Further, a pair of strong rigidity portions 608a and 609a are joined to the front and back surfaces of the FPC 601 to form a link 614a having rigidity, and a pair of strong rigidity portions 608b and 609b are joined to the front and back surfaces of the FPC 601 to form a link 614b having rigidity. Then, the link 611 and the link 612, the link 612 and the link 613, the link 613 and the link 614a, and the link 614b and the link 611 constitute hinge parts 621, 622, 623, and 624 connected by the FPC 601.

In the open link structure body 600, the link 611 has an electrode pad 631 for electrical connection or signal extraction by providing the strong rigidity portion 603 with an opening in a center and exposing a conductive layer of the FPC 601 to the outside through the opening, the link 612 has an electrode pad 632 for electrical connection or signal extraction by providing the strong rigidity portion 605 with an opening in a center and exposing a conductive layer of the FPC 601 to the outside through the opening, and the link 613 has an electrode pad 633 for electrical connection or signal extraction by providing the strong rigidity portion 607 with an opening in a center and exposing a conductive layer of the FPC 601 to the outside through the opening. Furthermore, electrode pads 601a and 601b are provided at respective ends of links 614a and 614b at both ends of the open link structure body 600.

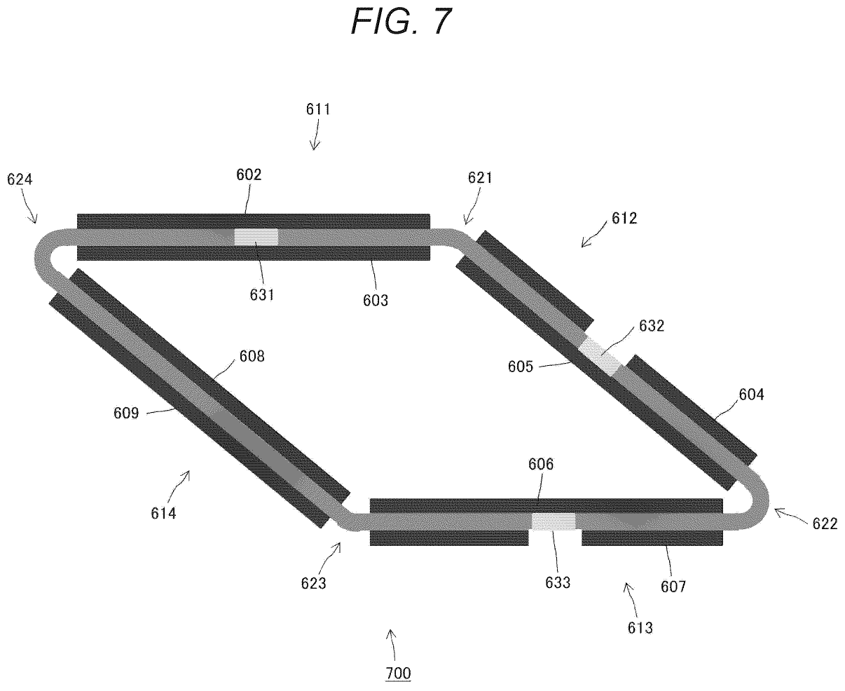

FIG. 7 illustrates an example of a closed link structure body 700 configured using an FPC. In the illustrated closed link structure body 700, the FPC 601 constituting the open link structure body 600 illustrated in FIG. 6 is bent, and the links 614a and 614b at both ends are joined to the provided electrode pads 601a and 601b, respectively, to constitute the closed link structure. Then, the joined links 614a and 614b are newly defined as a link 614.

In the closed link structure body 700, since lengths of the facing link 611 and link 613 are equal to each other and lengths of the facing link 612 and link 614 are equal to each other, a parallel link mechanism (alternatively, a four-node linkage mechanism) can be configured. Therefore, when a driving link moves, a driven link makes the same movement, and an angle of facing links is always maintained.

In the closed link structure body 700, each of the hinge parts 621, 622, 623, and 624 includes only the FPC and has a spring element in a rotation direction, and thus constitutes a flexible joint. In other words, each of the hinge parts 621, 622, 623, and 624 includes only the FPC, in other words, the conductive layer passes through a rotation axis, so that a wiring structure passing inside the hinge is achieved. Even if a rotational operation occurs between links, stress such as a tension and a compression force affecting conductivity is suppressed to be low, so that a risk of an adverse effect on control performance and cutting of wiring is extremely low.

By combining such a closed link structure body 700, it is possible to configure a surgical robot that is mounted with a surgical tool and mechanically achieves a pivot movement as illustrated in FIG. 3.

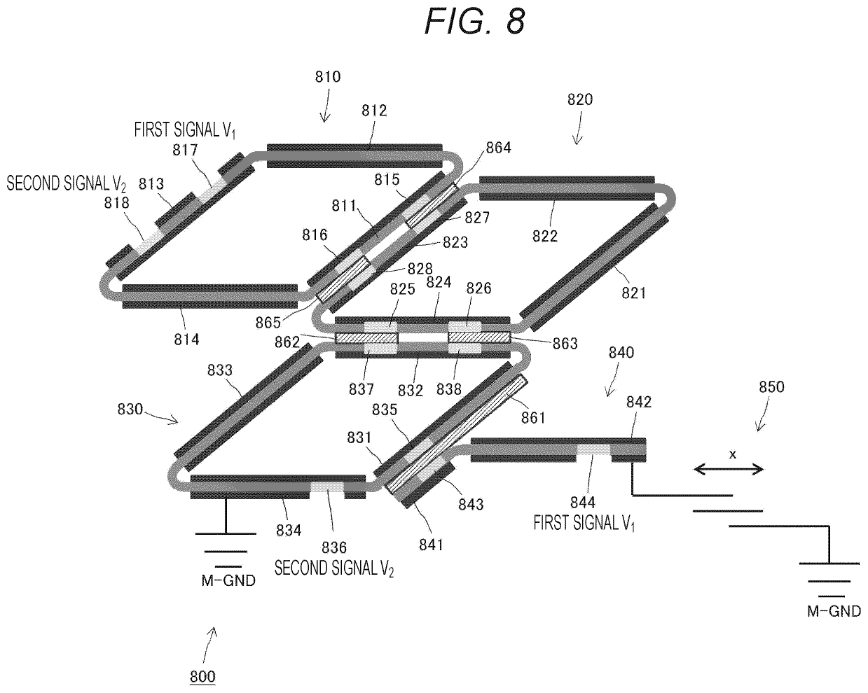

FIG. 8 illustrates a configuration example of a surgical robot 800 that achieves a pivot movement by connecting a plurality of closed link structure bodies using an FPC as illustrated in FIG. 7. The surgical robot 800 basically has the same degree of freedom as the robot 300 illustrated in FIG. 3.

In the surgical robot 800, a closed link structure body 810, a closed link structure body 820, and a closed link structure body 830 are connected in this order from a distal end. Moreover, an open link structure body 840 having a linear motion mechanism is connected to the closed link structure body 830. Note that, specific configurations of the individual closed link structure bodies 810 to 830 and the open link structure body 840 are similar to those illustrated in FIGS. 5 and 6, and thus, a detailed description thereof is omitted here.

One link 834 of the closed link structure body 830 on a proximal end side serves as a mechanical ground (alternatively, a fixed link). To a link 831 hinge-connected to one end of the link 834, a link 841 of the open link structure body 840 is connected. Furthermore, a link 842 of the open link structure body 840 can be displaced in a horizontal direction (or an x direction) in a plane of the figure, by a linear motion actuator 850 having one end serving as a mechanical ground. Therefore, the link 831 is a driving link. Furthermore, a link 833 facing the link 831 is a driven link, and another link 832 is an intermediate link.

The open link structure body 840 has an electrode pad 843 at one location of the link 842 and an electrode pad 844 at one location of the link 841. The electrode pad 843 is used to input and output a first signal V₁, and the electrode pad 844 is used to transmit the first signal V₁ between with the closed link structure body 830 side.

The link 831 of the closed link structure body 830 has an electrode pad 835 of one location at a position facing the electrode pad 844. Then, the link 841 of the open link structure body 840 is fixed to the link 831 on the closed link structure body 830 side while ensuring conductivity of the electrode pad 844 and the electrode pad 835 via a conductive joint part 961. Therefore, the closed link structure body 830 is capable of transmitting the first signal V₁ between with the open link structure body 840. Furthermore, the closed link structure body 830 has an electrode pad 836 at one location of the link 834. The electrode pad 836 is used to input and output a second signal V₂.

The closed link structure body 830 has electrode pads 837 and 838 for the first signal and the second signal, respectively, at two locations of the link 832. Furthermore, a link 824 connected to the link 832 on the closed link structure body 820 side has electrode pads 825 and 826 of two locations at positions facing the electrode pads 837 and 838 each. Then, the link 824 is fixed to the link 832 while ensuring conductivity between the electrode pad 825 and the electrode pad 837 and between the electrode pad 826 and the electrode pad 838 via joint parts 862 and 863 having conductivity, respectively. Therefore, the first signal V₁ and the second signal V₂ can be transmitted between the closed link structure body 830 and the closed link structure body 820.

The closed link structure body 820 has electrode pads 827 and 828 for the first signal V₁ and the second signal V₂, respectively, at two locations of a link 823. Furthermore, a link 811 located on the closed link structure body 810 side and connected to the link 823 has electrode pads 815 and 816 of two locations at positions facing the electrode pads 827 and 828 each. Then, the link 811 is fixed to a link 822 while ensuring conductivity between the electrode pad 815 and the electrode pad 827 and between the electrode pad 816 and the electrode pad 828 via joint parts 864 and 865 having conductivity, respectively. Therefore, the first signal V₁ and the second signal V₂ can be transmitted between the closed link structure body 820 and the closed link structure body 810.

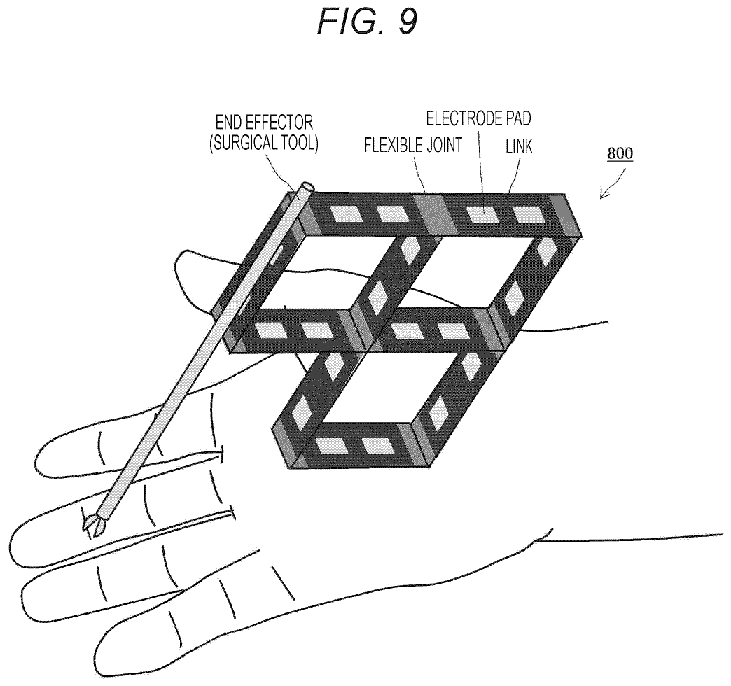

A link 813 of the closed link structure body 811 corresponds to a link at a distal end of the surgical robot 800, and constitutes a mounting part of an end effector (not illustrated in FIG. 9) including a surgical tool such as a forceps. Then, at two locations of the link 813, electrode pads 817 and 818 for the first signal V₁ and the second signal V₂ are provided, respectively. Therefore, the surgical robot 800 can transmit the first signal V₁ and the second signal V₂ between with the end effector mounted at the distal end.

The surgical tool used by being mounted to the surgical robot 800 holds a memory that stores, for example, a surgical tool identification ID for identifying a type, a specification, performance, or individual information of the surgical tool, authentication information for determining usage on the surgical robot 800, calibration data at a time of operating the surgical tool, and the like. Then, the surgical robot 800 can access the surgical tool through an electrical interface including the electrode pads 817 and 818 at the distal end, read the surgical tool identification ID from the memory, and transmit the corresponding authentication information, calibration data, and the like to the memory in the surgical tool.

The surgical robot 800 according to the present embodiment has a wiring structure in which a signal line used for transmission of the first signal V₁ and the second signal V₂ passes inside the hinge. Therefore, even if the surgical robot 800 operates and a rotational operation occurs between links, stress such as a tension and a compression force affecting conductivity is suppressed to be low, so that a risk of an adverse effect on control performance and cutting of wiring is extremely low.

On a signal transmission path, a control signal and electric power to the surgical tool which is the end effector, a signal of information read from the memory in the surgical tool, and the like are transmitted. Note that FIG. 8 has illustrated an example in which the surgical robot 800 has a signal transmission path including two bits of the first signal V₁ and the second signal V₂, but a bit width of the signal transmission path can be easily extended to three bits or more.

In FIG. 8, the description has been made with a plan view of the surgical robot 800 viewed from just beside, in which each link is drawn like a wire material, for convenience of description. In practice, the link uses the FPC as a base material, and thus is a rigid body having a constant width. FIG. 9 illustrates an example of a three-dimensional image of the surgical robot 800 including a closed link structure body using an FPC.

FIG. 9 illustrates a state where an end effector including a surgical tool such as a forceps is attached to a link at a distal end of the surgical robot 800. Since the link and the joint are configured using the FPC, it is easy to manufacture the link and the joint in a small size for ophthalmic surgery. For example, the surgical robot 800 that is small enough to be placed on a palm can be manufactured at a relatively low cost. Wiring can be easily routed from the mounting part of the end effector to the mechanical ground. In particular, by omitting aerial wiring around the end effector, separation of a clean region and a non-clean region and cleaning/sterilization work are facilitated. Furthermore, by boring an opening in a strong rigidity portion to be attached to a link portion, it is possible to provide an electrode pad for inputting and outputting an electric signal at any position of a manipulator, so that a degree of freedom of mechanical design is improved.

Note that the multi-link structure body using the electric circuit board as described in this Paragraph E is merely an example, and it is possible to manufacture a robot including a flexible joint without using such a multi-link structure body.

### INDUSTRIAL APPLICABILITY

The present disclosure is heretofore described in detail with reference to the specific embodiment. However, it is obvious that those skilled in the art can make modifications and substitutions of the embodiment without departing from the gist of the present disclosure.

The present disclosure can be mainly applied to ophthalmic surgery such as fundus surgery, but can also be similarly applied to various surgeries performed by inserting a surgical tool into a body via a trocar. Furthermore, the present disclosure can also be applied to, for example, remote control or operation assist using a master-slave robot, and autonomous control of a surgical robot.

Furthermore, the surgical tool mounted to the surgical robot according to the present disclosure may be a forceps, tweezers, an insufflation tube, an energy treatment tool, or a medical observation device such as a microscope, or an endoscope (rigid endoscopes such as a laparoscope and an arthroscope, and soft endoscopes such as a gastrointestinal endoscope and a bronchoscope).

In short, the present disclosure is heretofore described in a form of an example and the content described in this specification should not be interpreted in a limited manner. In order to determine the gist of the present disclosure, the claims should be taken into consideration.

Note that the present disclosure can have the following configurations.
(1) A surgical robot configured by a plurality of links and to be mounted with a surgical tool, in which
at least some links connecting between links are formed by a flexible joint having flexibility.
(2) The surgical robot according to (1) described above, in which
the flexible joint has a spring element in a rotation direction.
(3) The surgical robot according to (2) described above, in which
the spring element is any of polyimide, CFRP, GFRP, and polyester.
(4) The surgical robot according to any one of (1) to (3) described above, in which
to the flexible joint, a flexible structure serially fixed to an output shaft of a driving motor is connected.
(5) The surgical robot according to any one of (1) to (4) described above, in which
a surgical tool for ophthalmic surgery is mounted.

### REFERENCE SIGNS LIST

100 Surgical robot
101 Base part
102 Link
103 Joint
104, 106, 108, 110 Link
105, 107, 109 Flexible joint
111 Surgical tool
201, 202 Trocar
203 Surgical tool

1. A surgical robot configured by a plurality of links and to be mounted with a surgical tool, wherein
at least some links connecting between links are formed by a flexible joint having flexibility.

2. The surgical robot according to claim 1, wherein
the flexible joint has a spring element in a rotation direction.

3. The surgical robot according to claim 2, wherein
the spring element is any of polyimide, carbon fiber reinforced plastic (CFRP), glass fiber reinforced plastic (GFRP), and polyester.

4. The surgical robot according to claim 1, wherein
to the flexible joint, a flexible structure serially fixed to an output shaft of a driving motor is connected.

5. The surgical robot according to claim 1, wherein
a surgical tool for ophthalmic surgery is mounted.
